(19) United States
(12) Patent Application Publication
(10) Pub. No.: US 2024/0180946 A1
(43) Pub. Date: Jun. 6, 2024

(54) COMPOSITIONS COMPRISING TOPIRAMATE FOR TREATING DERMATOLOGICAL CONDITIONS

(71) Applicants: Sabina GLOZMAN, Naharya (IL); Orgenesis Inc., Germantown, MD (US)

(72) Inventors: SABINA GLOZMAN, NAHARYA (IL); ITZHAK VIDER, TEL-AVIV (IL); VERED CAPLAN, COPPET (CH)

(73) Assignee: Orgenesis Inc., Germantown, MD (US)

(21) Appl. No.: 18/552,139

(22) PCT Filed: Mar. 24, 2022
(86) PCT No.: PCT/US2022/021650

§ 371 Date: Sep. 22, 2023

Related U.S. Application Data
(60) Provisional application No. 63/165,594, filed on Mar. 24, 2021.

(51) Int. Cl. A61K 31/7048; A61K 9/00; A61K 9/50; A61K 31/728; A61K 45/06; A61P 17/18

(52) U.S. Cl. CPC A61K 31/7048; A61K 9/0014; A61K 9/5063; A61K 31/728; A61K 45/06; A61P 17/18

(57) ABSTRACT

Disclosed herein are compositions comprising topiramate and bioxome, redoxome, HA, extracellular vesicles (EV), or PRP extracellular vesicles, or any combination thereof. Further provided herein are methods for treating dermatological conditions, and methods for testing said compositions.

### BACKGROUND OF THE INVENTION

Skin is the barrier that segregates the body from the outer environment. Besides protecting the body from water loss and microorganism infection, it has an important cosmetic role. Young and beautiful appearance may have a positive influence on people's social behavior and reproductive status. However, aging of organs begins from the time when one is born, and there is no exception for the skin. As the most voluminous organ of the body, the skin shows obvious and visible sign of aging when one becomes older. Therefore, for many people, especially females, a considerable amount of daily expense is occupied by cosmetics and pharmaceuticals attempting to prevent or reverse skin aging. Skin aging is a part of a natural human “aging mosaic” which becomes evident and follows different trajectories in different organs, tissues and cells with time. While the aging signs of internal organs are masked from the ambient “eyes,” the skin provides first obvious marks of the passing time. This vast cosmetic need continually promotes research on skin aging and its treatment. Different models are proposed to explain the molecular basis for skin aging, including the theory of cellular senescence, decrease in cellular DNA repair capacity and loss of telomeres, point mutations of extranuclear mitochondrial DNA, oxidative stress, increased frequency of chromosomal abnormalities, single-gene mutations, reduced sugar, chronic inflammation, and so on.

In 2018, the global anti-aging market was estimated to be worth about 50.2 billion U.S. dollars. The anti-aging market is estimated to see a compound annual growth rate (CAGR) of 5.7 percent between 2018 and 2023.

Several anti-aging strategies have been developed and used, and include preventive measurements, cosmetological strategies, topical and systemic therapeutic agents and invasive procedures.

Psoriasis is a chronic inflammatory skin condition characterized by clearly defined, red and scaly plaques (thickened skin). It is classified into several subtypes. Psoriasis affects 2-4% of males and females. It can start at any age including childhood, with peaks of onset at 15-25 years and 50-60 years. It tends to persist lifelong, fluctuating in extent and severity. It is particularly common in caucasians but may affect people of any race. About one-third of patients with psoriasis have family members with psoriasis.

No cure for psoriasis is known, but various treatments can help control the symptoms. Current treatments include topical treatment, phototherapy and system treatment (including immunosuppressive drugs and biologics). The global psoriasis drugs market size is expected to be valued at USD 21.11 billion by 2022.

Accordingly, there is a need to develop compositions with enhances therapeutic effects, and to use these compositions for treating skin aging and psoriasis as well as other dermatological conditions that are currently without satisfactory treatment.

The present disclosure teaches a composition comprising topiramate and bioxome, redoxome, hyaluronic acid (HA), extracellular vesicles (EV), or platelet-derived (PRP) extracellular vesicles, or any combination thereof and methods for using these compositions in the treatment of dermatological compositions including akin aging and psoriasis.

### SUMMARY OF THE INVENTION

Disclosed herein in one aspect, is a composition comprising topiramate and bioxome, redoxome, hyaluronic acid (HA), extracellular vesicles (EV), or platelet rich derived (PRP) extracellular vesicles, or any combination thereof. In a related aspect, the composition comprises topiramate and a bioxome. In a related aspect, the topiramate is encapsulated by the bioxome.

In a related aspect, the composition further comprises an excipient. In a related aspect, at least one excipient, or topiramate, or any combination thereof are encapsulated in said bioxome, in said redoxome, in said HA, in said extracellular vesicles (EV), or in said PRP extracellular vesicles.

In a related aspect, the composition is formulated for topical delivery. In a related aspect, when the composition is formulated for topical delivery, the excipient is selected from the group comprising: C12-C15 alkyl benzoate, mineral oil, glycerin, cetearyl alcohol, glyceryl stearate SE, dimethicone, steareth-2, steareth-21, aloe barbadensis leaf juice, panthenol, disodium EDTA, acrylates/C10-30 alkyl acrylate crosspolymer, tocopheryl acetate, retinyl palmitate, hydroxypropyl cyclodextrin, palmitoyl tripeptide 38, hydroxyethilcellulose, sodium hydroxide, phenoxyethanol, caprylyl glycol, or any combination thereof.

In a related aspect, the composition further comprises an additional therapeutic agent. In a related aspect, the additional therapeutic agent is a therapeutic agent for healing inflammatory condition of the skin.

Disclosed herein in one aspect, a method for treating a dermatological condition comprising a step of administering to a subject in need thereof a composition comprising topiramate and bioxome, redoxome, HA, extracellular vesicles (EV), or PRP extracellular vesicles, or any combination thereof. In a related aspect, the composition comprises topiramate and a bioxome. In a related aspect, the dermatological condition is an inflammatory condition. In a related aspect, the inflammatory condition is a TNF mediated inflammatory disease. In a related aspect, the TNF mediated inflammatory disease is selected from akin aging or psoriasis.

In a related aspect, the skin aging comprises a combination of at least two of cellular inflammation, oxidative stress, senescence, autophagy disbalance, telomerase deactivation and epigenetic. In a related aspect, the psoriasis comprises at least one of loss of differentiation balance, immune overproliferation of keratinocytes and inflammation skin disorders.

In a related aspect, the method described above comprises administering a composition further comprising an additional therapeutic agent for healing an inflammatory condition of the skin.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a graphical representation of epidermal viability of the ex vivo organ cultures of human skin.

FIG. 2 is a graphical representation of anti-inflammatory effect of Bioxome™-encapsulated Topiramate in the ex vivo human skin inflammation model.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present compositions and methods of use thereof may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure. Similarly, it is to be understood that the embodiments disclosed herein are combinable.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

A skilled artisan would appreciate that the term “comprising” encompasses inclusion of the recited elements, but not excluding others which may be optional. For example, but not limited to a composition comprising topiramate and autologous cells, which can comprise elements other than topiramate and autologous cells.

A skilled artisan would understand that when a range of values is expressed, another embodiment includes one limit value and/or the other limit value of the range. For example, a composition comprising from about 2×10⁵ to 5×10⁵ autologous cells per ml, comprises an embodiment comprising 2×10⁵ and/or 5×10⁵ autologous cells per ml. Additionally, all ranges are inclusive and combinable.

### Compositions and Delivery Systems

In some embodiments, the present invention relates to a composition comprising topiramate and bioxome, redoxome, Hyaluronic acid (HA), extracellular vesicles (EV), or platelet-derived (PRP) extracellular vesicles, or any combination thereof.

Bioxomes may refer to artificial, submicron nano-particles having resemblance to natural extracellular vesicles (EV). In one embodiment, the bioxome particle is carrying a cargo comprising at least one active molecule. In another embodiment the cargo comprises at least two active molecules. In another embodiment, the cargo comprises a plurality of active molecules.

Redoxomes may refer to bioxome particles carrying a cargo comprising at least one redox active, free radical scavenging compound.

In one embodiment, the redoxome comprises fenton reaction complex blockers, hydroxyl radical trap, iron chelator and a lipid radical trap. In one embodiment, the radical trap is ascorbic acid, nitric oxid donor (S-nitrosoglutathione) , or a derivative thereof. A non-limiting list of iron chelators of the invention comprises, without limitation, des ferrioxamine (DFX), ethylenediaminetetraacetic acid (EDTA), rutin, disodium EDTA, tetrasodium EDTA, calcium disodium EDTA, diethylenetriaminepentaacetic acid (DTPA) or a salt thereof, hydroxyethlethylenediaminetriacetic acid (HEDTA) or a salt thereof, nitrilotriacetic acid (NTA), acetyl trihexyl citrate, aminotrimethylene phosphonic acid, beta-alanine diacetic acid, bismuth citrate, citric acid, cyclohexanediamine tetraacetic acid, diammonium citrate, dibutyl oxalate, diethyl oxalate, diisobutyl oxalate, diisopropyl oxalate, dilithium oxalate, dimethyl oxalate, dipotassium EDTA, dipotassium oxalate, dipropyl oxalate, disodium EDTA-copper, disodium pyrophosphate, etidronic acid, HEDTA, methyl cyclodextrin, oxalic acid, pentapotassium, triphosphate, pentasodium aminotrimethylene phosphonate, pentasodium pentetate, pentasodium triphosphate, pentetic acid, phytic acid, potassium citrate, sodium citrate, sodium dihydroxyethylglycinate, sodium gluceptate, sodium gluconate, sodium hexametaphosphate, sodium metaphosphate, sodium metasilicate, sodium oxalate, sodium trimetaphosphate, tea-EDTA, tetrahydroxypropyl ethylenediamine, tetrapotassium etidronate, tetrapotassium pyrophosphate, tetrasodium etidronate, tetrasodium pyrophosphate, tripotassium EDTA, trisodium EDTA, trisodium hedta, trisodium NTA, trisodium phosphate, malic acid, fumaric acid, maltol, succimer, penicillamine, dimercaprol, deferipron, a natural protein based iron chelator, melatonin, siderphore, zinc or copper cation, or salt or complex, and desferrioxamine mesylate, or a combination thereof. In another embodiment, the iron chelator is selected from the group consisting of EDTA (ethylenediaminetetraacetic acid), DTPA (diethylene triamine pentaacetic acid), NTA (nitrilotriacetic acid), detoxamin, deferoxamine, deferiprone, deferasirox, glutathione, metalloprotein, ferrochel (bis-glycinate chelate), ceruloplasmin, penicillamine, cuprizone, trientine, ferrulic acid, zinc acetate, lipocalin 2, and dimercaprol.

Hyaluronic acid (HA), also called hyaluronan, is an anionic, nonsulfated glycosaminoglycan present in connective, epithelial, and neural tissues. HA is one of the main components of the extracellular matrix and contributes significantly to cell proliferation and migration.

HA might play an important role in wound healing and has been used in the synthesis of biological scaffolds for wound-healing applications. HA roles in wound healing might be related to its several functions during wound-related inflammation. In some embodiments, HA promotes early inflammation in the wound environments. In some embodiments, HA moderates the inflammatory response, thus stabilizing granulation tissue matrix. In some embodiments, HA promotes a conducive environment for migration of cells into the wound matrix. In some embodiments, HA promotes the reepithelization process. In some embodiments HA scavenges free-radicals. In some embodiments, HA promotes keratinocyte proliferation and migration.

In some embodiments, HA comprises a new HA specifically designed for the composition. In some embodiments, the new HA has specific and unique characteristics for optimal combination with autologous cells. In some embodiments, HA facilitates autologous cell engraftment.

Extracellular vehicles (EVs) may refer to a heterogeneous collection of membrane-bound carriers with complex cargoes including proteins, lipids, and nucleic acids. They are known to be released by all cell types and can be taken up by other cells, leading to the transfer of the cargo they carry. As such, they represent an important type of intercellular signaling and a natural mechanism for transferring macromolecules between cells. This ability to transfer cargo could be harnessed to deliver therapeutic molecules.

PRP extracellular vehicles (platelet-derived ECs) may refer to the most abundant EVs in human blood. PRP extracellular vehicles have been known to be effective in promoting tissue repair.

In one embodiment, the composition comprises topiramate and a bioxome. In one embodiment, the composition comprises topiramate and a redoxome. In one embodiment, the composition comprises topiramate and a HA. In one embodiment, the composition comprises topiramate and an extracellular vesicles (EV). In one embodiment, the composition comprises topiramate and a PRP extracellular vesicle. In another embodiment, the composition comprises topiramate, bioxome and redoxome. In another embodiment, the composition comprises topiramate, bioxome and HA. In another embodiment, the composition comprises topiramate, bioxome and extracellular vesicles (EV). In another embodiment, the composition comprises topiramate, bioxome and PRP extracellular vesicles. In another embodiment, the composition comprises topiramate, redoxome and HA. In another embodiment, the composition comprises topiramate, redoxome and extracellular vesicles (EV). In another embodiment, the composition comprises topiramate, redoxome and PRP extracellular vesicles. In another embodiment, the composition comprises topiramate, HA and extracellular vesicles (EV). In another embodiment, the composition comprises topiramate, HA and PRP extracellular vesicles. In another embodiment, the composition comprises topiramate, extracellular vesicles (EV) and PRP extracellular vesicles. In another embodiment, the composition comprises topiramate, bioxome, redoxome and HA. In another embodiment, the composition comprises topiramate, bioxome, redoxome and extracellular vesicles (EV). In another embodiment, the composition comprises topiramate, bioxome, redoxome and PRP extracellular vesicles. In another embodiment, the composition comprises topiramate, bioxome, HA and extracellular vesicles (EV). In another embodiment, the composition comprises topiramate, bioxome, HA and PRP extracellular vesicles.

In one embodiment, the composition further comprises an excipient. In another embodiment, the excipient is selected from the group comprising: alpha-tocopherol, ascorbic acid, benzoic acid, castor oil, cholesterol, corn oil, cottonseed oil, creatine, creatinine, dextrose solution, gelatin, gentisic acid, gentisic acid ethanolamide, glucuronic acid, glycerin, lactic acid, hydrogenated soybean lecithin, niacinamide, caprylic acid, oleic acid, palmitic acid, peanut oil, PEG vegetable oil, PEG-40 castor oil, PEG-60 hydrogenated castor oil, phenylalanine, phospholipid, polyoxyl 35 castor oil, sesame oil, soybean oil, urea, zinc oxide, or any combination thereof.

In another embodiment, the excipient is selected from the group comprising: C12-C15 alkyl benzoate, mineral oil, glycerin, cetearyl alcohol, glyceryl stearate SE, dimethicone, steareth-2, steareth-21, aloe barbadensis leaf juice, panthenol, disodium EDTA, acrylates/C10-30 alkyl acrylate crosspolymer, tocopheryl acetate, retinyl palmitate, hydroxypropyl cyclodextrin, palmitoyl tripeptide 38, hydroxyethilcellulose, sodium hydroxide, phenoxyethanol, caprylyl glycol, or any combination thereof.

In one embodiment, at least one excipient, or topiramate, or combination thereof are encapsulated in said bioxome, in said redoxome, in said HA, in said extracellular vesicles (EV), or in said PRP extracellular vesicles. In another embodiment, the topiramate is encapsulated in said bioxome. In another embodiment, the topiramate is encapsulated in said redoxome. In another embodiment, the topiramate is encapsulated in said HA. In another embodiment, the topiramate is encapsulated in said extracellular vesicles (EV). In another embodiment, the topiramate is encapsulated in said PRP extracellular vesicles. In another embodiment, at least one excipient is encapsulated in said bioxome. In another embodiment, at least one excipient is encapsulated in said redoxome. In another embodiment, at least one excipient is encapsulated in said HA. In another embodiment, at least one excipient is encapsulated in said extracellular vesicles (EV). In another embodiment, at least one excipient is encapsulated in said PRP extracellular vesicles.

In one embodiment, the topiramate is dissolved in Hexane-Isopropanol (HIP) together with lipids. In another embodiment, the use of HIP and lipids in the composition improves the incorporation of topiramate into the bioxome membrane. In another embodiment, the use of HIP and lipids in the composition improved the anti-inflammatory effect.

In one embodiment, the composition is formulated for topical delivery.

In one embodiment, the composition further comprises an additional therapeutic agent. In another embodiment, the additional therapeutic agent is a therapeutic agent for healing inflammatory condition of the skin. In another embodiment, the additional therapeutic agent comprises Non Steroidal Anti Inflammatory Drugs (NSAIDs), antileukotrienes, Immune Selective Anti-Inflammatory Derivatives (ImSAIDs), anti TNFalpha drugs, or any combination thereof. In one embodiment, the therapeutic agent for healing an inflammatory condition of the skin comprises a Non Steroidal Anti Inflammatory Drug (NSAID). In one embodiment, the therapeutic agent for healing an inflammatory condition of the skin comprises an antileukotriene. In one embodiment, the therapeutic agent for healing an inflammatory condition of the skin comprises an Immune Selective Anti-Inflammatory Derivative (ImSAID). In one embodiment, the therapeutic agent for healing an inflammatory condition of the skin comprises an anti TNFalpha drug.

A skilled artisan would appreciate that Non Steroidal Anti Inflammatory Drugs (NSAIDs) are compounds that alleviate pain and inflammation by counteracting the cyclooxygenase (COX) enzyme. The NSAIDs prevent the prostaglandins from ever being synthesized, reducing or eliminating the inflammation and resulting pain. In one embodiment, the NSAIDs comprises acetylsalicylic acid (ASA), paracetamol, ibuprofen or naproxen.

A skilled artisan would appreciate that antileukotrienes are anti-inflammatory agents which function as leukotriene-related enzyme inhibitors (arachidonate 5-lipoxygenase) or leukotriene receptor antagonists (cysteinyl leukotriene receptors) and consequently oppose the function of these inflammatory mediators.

A skilled artisan would appreciate that Immune Selective Anti-Inflammatory Derivatives (ImSAIDs) are a class of peptides that have anti-inflammatory properties. ImSAIDs work by altering the activation and migration of inflammatory cells, which are immune cells responsible for amplifying the inflammatory response.

A skilled artisan would appreciate that anti TNFalpha drugs suppresses the physiologic response to tumor necrosis factor (TNF), which is part of the inflammatory response. In one embodiment, the anti TNF alpha compounds comprises infliximab, adalimumab, certolizumab pegol, golimumab, etanercept, thalidomide, lenalidomide, pomalidomide, pentoxifylline, or bupropion.

### Method for Treating Dermatological Conditions

In some embodiments, the present invention relates to a method for treating a dermatological condition, the method comprising a step of administering to a subject in need thereof a composition comprising topiramate and bioxome, redoxome, HA, extracellular vesicles (EV), or PRP extracellular vesicles, or any combination thereof.

In one embodiment, the method comprising a step of administering a composition comprising topiramate and a bioxome. In another embodiment, the method comprising a step of administering a composition comprising topiramate and a redoxome. In another embodiment, the method comprising a step of administering a composition comprising topiramate and a HA. In another embodiment, the method comprising a step of administering a composition comprising topiramate and an extracellular vesicles (EV). In another embodiment, the method comprising a step of administering a composition comprising topiramate and a PRP extracellular vesicle. In another embodiment, the method comprising a step of administering a composition comprising topiramate, bioxome and redoxome. In another embodiment, the method comprising a step of administering a composition comprising topiramate, bioxome and HA. In another embodiment, the method comprising a step of administering a composition comprising topiramate, bioxome and extracellular vesicles (EV). In another embodiment, the method comprising a step of administering a composition comprising topiramate, bioxome and PRP extracellular vesicles. In another embodiment, the method comprising a step of administering a composition comprising topiramate, redoxome and HA. In another embodiment, the method comprising a step of administering a composition comprising topiramate, redoxome and extracellular vesicles (EV). In another embodiment, the method comprising a step of administering a composition comprising topiramate, redoxome and PRP extracellular vesicles. In another embodiment, the method comprising a step of administering a composition comprising topiramate, HA and extracellular vesicles (EV). In another embodiment, the method comprising a step of administering a composition comprising topiramate, HA and PRP extracellular vesicles. In another embodiment, the method comprising a step of administering a composition comprising topiramate, extracellular vesicles (EV) and PRP extracellular vesicles. In another embodiment, the method comprising a step of administering a composition comprising topiramate, bioxome, redoxome and HA. In another embodiment, the method comprising a step of administering a composition comprising topiramate, bioxome, redoxome and extracellular vesicles (EV). In another embodiment, the method comprising a step of administering a composition comprising topiramate, bioxome, redoxome and PRP extracellular vesicles. In another embodiment, the method comprising a step of administering a composition comprising topiramate, bioxome, HA and extracellular vesicles (EV). In another embodiment, the method comprising a step of administering a composition comprising topiramate, bioxome, HA and PRP extracellular vesicles.

In one embodiment, the invention relates to a method of treating dermatological condition, wherein the dermatological condition is an inflammatory condition. In another embodiment, the inflammatory condition is a TNF mediated inflammatory disease. In another embodiment, the TNF mediated inflammatory disease is selected from rheumatoid arthritis, psoriatic arthritis, psoriasis, axial spondyloarthropathies, crohn's disease, ulcerative colitis, juvenile idiopathic arthritis, ankylosing spondylitis (AS), type 1 diabetes, inflammatory bowel disease (IBD), allergy, Behcet's disease, skin aging or multiple sclerosis. In another embodiment, the TNF mediated inflammatory disease is selected from skin aging or psoriasis. In another embodiment, the inflammatory condition may be skin aging. In another embodiment, the inflammatory condition may be psoriasis. In another embodiment, the psoriasis is selected from plaque psoriasis, guttate psoriasis, pustular psoriasis, inverse psoriasis, erythrodermic psoriasis, nail psoriasis, psoriatic arthritis, or any combination thereof.

In one embodiment, the invention relates to a method of treating skin aging, wherein the skin aging comprises a combination of at least two of, cellular inflammation, oxidative stress, senescence, autophagy disbalance, telomerase deactivation and epigenetic. In another embodiment, the invention relates to a method of treating skin aging, wherein the skin aging comprises a combination of cellular inflammation and oxidative stress. In another embodiment, the invention relates to a method of treating skin aging, wherein the skin aging comprises a combination of cellular inflammation and senescence. In another embodiment, the invention relates to a method of treating skin aging, wherein the skin aging comprises a combination of cellular inflammation and autophagy disbalance. In another embodiment, the invention relates to a method of treating skin aging, wherein the skin aging comprises a combination of cellular inflammation and telomerase deactivation. In another embodiment, the invention relates to a method of treating skin aging, wherein the skin aging comprises a combination of cellular inflammation and epigenetic. In another embodiment, the invention relates to a method of treating skin aging, wherein the skin aging comprises a combination of oxidative stress and senescence. In another embodiment, the invention relates to a method of treating skin aging, wherein the skin aging comprises a combination of oxidative stress and autophagy disbalance. In another embodiment, the invention relates to a method of treating skin aging, wherein the skin aging comprises a combination of oxidative stress and telomerase deactivation. In another embodiment, the invention relates to a method of treating skin aging, wherein the skin aging comprises a combination of oxidative stress and epigenetic. In another embodiment, the invention relates to a method of treating skin aging, wherein the skin aging comprises a combination of senescence and autophagy disbalance. In another embodiment, the invention relates to a method of treating skin aging, wherein the skin aging comprises a combination of senescence and telomerase deactivation. In another embodiment, the invention relates to a method of treating skin aging, wherein the skin aging comprises a combination of senescence and epigenetic. In another embodiment, the invention relates to a method of treating skin aging, wherein the skin aging comprises a combination of autophagy disbalance and telomerase deactivation. In another embodiment, the invention relates to a method of treating skin aging, wherein the skin aging comprises a combination of autophagy disbalance and epigenetic. In another embodiment, the invention relates to a method of treating skin aging, wherein the skin aging comprises a combination of telomerase deactivation and epigenetic.

In one embodiment, the invention relates to a method of treating psoriasis, wherein the psoriasis comprises at least one of loss of differentiation balance, immune overproliferation of keratinocytes and inflammation skin disorders. In another embodiment, the invention relates to a method of treating psoriasis, wherein the psoriasis comprises loss of differentiation balance. In another embodiment, the invention relates to a method of treating psoriasis, wherein the psoriasis comprises immune overproliferation of keratinocytes. In another embodiment, the invention relates to a method of treating psoriasis, wherein the psoriasis comprises inflammation skin disorders.

In one embodiment, the method comprises a step of administering a composition wherein the composition further comprises an excipient. In another embodiment, the excipient is selected from the group comprising: alpha-tocopherol, ascorbic acid, benzoic acid, castor oil, cholesterol, corn oil, cottonseed oil, creatine, creatinine, dextrose solution, gelatin, gentisic acid, gentisic acid ethanolamide, glucuronic acid, glycerin, lactic acid, hydrogenated soybean lecithin, niacinamide, caprylic acid, oleic acid, palmitic acid, peanut oil, PEG vegetable oil, PEG-40 castor oil, PEG-60 hydrogenated castor oil, phenylalanine, phospholipid, polyoxyl 35, castor oil, sesame oil, soybean oil, urea, zinc oxide, or any combination thereof.

In another embodiment, the excipient is selected from the group comprising: C12-C15 alkyl benzoate, mineral oil, glycerin, cetearyl alcohol, glyceryl stearate SE, dimethicone, steareth-2, steareth-21, aloe barbadensis leaf juice, panthenol, disodium EDTA, acrylates/C10-30 alkyl acrylate crosspolymer, tocopheryl acetate, retinyl palmitate, hydroxypropyl cyclodextrin, palmitoyl tripeptide 38, hydroxyethilcellulose, sodium hydroxide, phenoxyethanol, caprylyl glycol, or any combination thereof.

In one embodiment, the method comprises a step of administering a composition wherein at least one excipient, or topiramate, or combination thereof are encapsulated in the bioxome, in the redoxome, in the HA, in the extracellular vesicles (EV), or in the PRP extracellular vesicles. In another embodiment, the topiramate is encapsulated in the bioxome. In another embodiment, the topiramate is encapsulated in the redoxome. In another embodiment, the topiramate is encapsulated in the HA. In another embodiment, the topiramate is encapsulated in the extracellular vesicles (EV). In another embodiment, the topiramate is encapsulated in the PRP extracellular vesicles. In another embodiment, at least one excipient is encapsulated in the bioxome. In another embodiment, at least one excipient is encapsulated in the redoxome. In another embodiment, at least one excipient is encapsulated in the HA. In another embodiment, at least one excipient is encapsulated in the extracellular vesicles (EV). In another embodiment, at least one excipient is encapsulated in the PRP extracellular vesicles.

In one embodiment, the method comprises administering the composition by topical administration. In one embodiment, the composition for topical administration comprises an excipient selected from the group comprising: C12-C15 alkyl benzoate, mineral oil, glycerin, cetearyl alcohol, glyceryl stearate SE, dimethicone, steareth-2, steareth-21, aloe barbadensis leaf juice, panthenol, disodium EDTA, acrylates/C10-30 alkyl acrylate crosspolymer, tocopheryl acetate, retinyl palmitate, hydroxypropyl cyclodextrin, palmitoyl tripeptide 38, hydroxyethilcellulose, sodium hydroxide, phenoxyethanol, caprylyl glycol, or any combination thereof.

In one embodiment, the method comprises administering an additional therapeutic agent. In another embodiment, the additional therapeutic agent is for healing an inflammatory condition of the skin. In one embodiment, the therapeutic agent for healing an inflammatory condition of the skin comprises Non Steroidal Anti Inflammatory Drugs (NSAIDs), antileukotrienes, Immune Selective Anti-Inflammatory Derivatives (ImSAIDs), anti TNFalpha drugs, or any combination thereof. In one embodiment, the therapeutic agent for healing an inflammatory condition of the skin comprises a Non Steroidal Anti Inflammatory Drug (NSAID). In one embodiment, the NSAIDs comprises acetylsalicylic acid (ASA), paracetamol, ibuprofen or naproxen. In one embodiment, the therapeutic agent for healing an inflammatory condition of the skin comprises an antileukotriene. In one embodiment, the therapeutic agent for healing an inflammatory condition of the skin comprises an Immune Selective Anti-Inflammatory Derivative (ImSAID). In one embodiment, the therapeutic agent for healing an inflammatory condition of the skin comprises an anti TNFalpha drug. In one embodiment, the anti TNF alpha compounds comprise infliximab, adalimumab, certolizumab pegol, golimumab, etanercept, thalidomide, lenalidomide, pomalidomide, pentoxifylline, or bupropion.

### Topiramate

Topiramate, also known as Topamax, Trokendi XR, Qudexy XR, and 2,3:4,5-bis-O-(1-methylethylidene)-β-D-fructopyranose sulfamate, is a molecule having Formula (I):

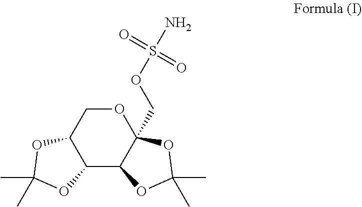

Topiramate has clinically demonstrated efficacy treating multiple dermatologic indications. Topiramate was shown to effectively treat acne scars, dermatitis, aging signs such as fine and deep wrinkles, and skin atrophy. Additionally, pre-clinical studies showed topiramate to significantly enhance wound healing. Topiramate therapeutic effect was observed when administered both orally as a tablet and topically as a cream.

In the current invention, the inventors describe surprising results for the efficacy of topiramate demonstrating superior anti-inflammatory effects when administered with bioxomes, in comparison with administration of topiramate alone.

### TNF

Tumor necrosis factor (TNF)-α has a key role in inflammatory responses that occur in the skin and has been identified as a key cytokine mediating cutaneous inflammation in the pathogenesis of several skin conditions including psoriasis. TNF-α plays a major role in initiating signaling pathways and pathophysiological responses after engaging TNF receptors.

In the skin, TNF-α is produced by fibroblasts, macrophages, monocytes and keratinocytes. TNF-α also plays an important role in photodamage and photoaging. Its secretion from keratinocytes (KCs) and dermal fibroblasts is specifically induced by UVB (290-320 nm), but not by UVA (320-400 nm), and it may therefore mediate harm from these more energetic UV wavelengths. For example, TNF-α released after UVB exposure induces nearby endothelial cells and KCs to display cell adhesion molecules, thereby recruiting inflammatory cells that secrete elastases and collagenases, leading to damage and aging of the skin. TNF-α also promotes apoptosis, lymphocyte activation, and hyperproliferative skin disorders. Besides UVB irradiation, several growth factors, and cytokines, including IL-1a, IL-1b, and IFNs also induce TNF-α expression in epidermal KCs.

Psoriasis is a T cell-mediated autoimmune disease. In its most prevalent form, psoriasis manifests as well-demarked erythemato-squamous plaques that may cover large areas of the patient's body. This form of psoriasis called plaque-type psoriasis is triggered by activated dermal dendritic cells that produce TNF and IL-23 and stimulate activation of autoimmune CD4+Th17 and CD8+Tc17 cells. Among the multiple effects of TNF-α on keratinocytes, the induction of matrix metalloproteinase-9 (MMP-9), a collagenase implicated in joint inflammation, might be one of the key mechanisms in psoriasis pathogenesis. MMP-9 expression can be enhanced also by osteopontin (OPN), a glycosylated protein whose levels are increased in skin and peripheral blood mononuclear cells (PBMC) of psoriasis patients. Anti-TNF-α treatment reduced MMP-9 expression in PBMC as well as lesioned skin of psoriatic patients. MMP-9 contributes to the chronic inflammatory process in the skin of the psoriatic patient either directly, by sustaining the inflammatory process and the tissue destruction, or indirectly, by allowing the traffic of inflammatory cells and enhancing the activity of inflammatory cytokines. MMPs can mediate the proteolytic process leading to the release of the soluble, active molecule of TNF-α from a cell membrane-anchored molecular form. Therefore, the inhibitory effect of anti-TNF-α therapy may thus offer a two-fold efficacy through both the reduction of MMP-9 levels and the inhibition of processing of the TNF-α precursor into its active molecular form.

The knowledge about the pathogenesis of plaque-type psoriasis has led to the development of therapies targeting the pathogenic cytokines, including anti-TNFs (etanercept, infliximab, adalimumab), anti-p40 (IL-12/IL-23, ustekinumab), and anti-IL-17A (secukinumab, ixekizumab). These antibodies now represent a benchmark for the treatment of moderate to severe psoriasis.

as indicated above, the pro-inflammatory cytokine TNF-α has also an important role in skin aging. TNF-α inhibits collagen synthesis and enhances collagen degradation by increasing the production of MMP-9. It also lowers the skin immunity and thus increases the risk of cutaneous infections in old age.

Three important factors affect skin aging. These are the natural process of aging (chronological aging), decreased estrogen levels, and harmful environmental factors like ultraviolet (UV) radiation.

As ultraviolet B (UVB) radiation promotes the production of TNF-α by dermal fibroblasts and epidermal keratinocytes, it increases inflammation. Moreover, macrophages that reside in the skin also secrete TNF-α.

### Pharmaceutical Compositions

The herein-described compositions can be incorporated into pharmaceutical compositions suitable for administration. In one embodiment, a “pharmaceutical composition” or a “pharmaceutical formulation” refers to a preparation of one or more of the active ingredients described herein with other chemical components such as physiologically suitable carriers and excipients. The purpose of a pharmaceutical composition or a “pharmaceutical formulation” is to facilitate administration of a compound to a subject. In certain embodiments, a “pharmaceutical composition” or a “pharmaceutical formulation” provides the pharmaceutical dosage form of a drug.

In some embodiments, “active ingredient” refers to the molecule which is accountable for the biological effect. In some embodiments, the active ingredient comprises topiramate.

In some embodiments, the pharmaceutical composition comprises an excipient. A skilled artisan would appreciate that, in some embodiments, excipients are selected according to the delivery mode of the pharmaceutical composition.

In some embodiments, when the pharmaceutical composition is formulated by topical delivery, the excipient is selected from the group comprising: hyaluronic acid (HA), C12-C15 alkyl benzoate, mineral oil, glycerin, cetearyl alcohol, glyceryl stearate SE, dimethicone, steareth-2, steareth-21, aloe barbadensis leaf juice, panthenol, disodium EDTA, acrylates/C10-30 alkyl acrylate crosspolymer, tocopheryl acetate, retinyl palmitate, hydroxypropyl cyclodextrin, palmitoyl tripeptide 38, hydroxyethilcellulose, sodium hydroxide, phenoxyethanol, caprylyl glycol, or any combination thereof.

Pharmaceutical compositions typically comprise a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference.

Some examples of substances which can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol (PEG); alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the compound is basically determined by the way the compound is to be administered. If the subject compound is to be injected, in one embodiment, the pharmaceutically-acceptable carrier is sterile, physiological saline, with a blood-compatible suspending agent, the pH of which has been adjusted to about 7.4.

In some embodiments, the pharmaceutical compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents(e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol (PEG), sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates), adjuvants, or any combination thereof.

Typical components of carriers for syrups, elixirs, emulsions and suspensions include ethanol, glycerol, propylene glycol, polyethylene glycol (PEG), liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. In another embodiment, peroral liquid compositions also contain one or more components such as sweeteners, flavoring agents and colorants disclosed above.

The compositions also include incorporation of the active component into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

In some embodiments, compositions including the preparation disclosed herein formulated in a compatible pharmaceutical carrier are also prepared, placed in an appropriate container, and labeled for treatment of an indicated condition.

In one embodiment, the pharmaceutical composition comprising topiramate and bioxome, redoxome, HA, extracellular vesicles (EV), or PRP extracellular vesicles, or any combination thereof, is stabilized when placed in buffered solutions having a pH between about 4 and 7.2. In another embodiment, the pharmaceutical composition comprising topiramate and bioxome, redoxome, HA, extracellular vesicles (EV), or PRP extracellular vesicles, or any combination thereof, is stabilized in a buffered solution having a pH between about 4 and 8.5. In another embodiment, the pharmaceutical composition comprising topiramate and bioxome, redoxome, HA, extracellular vesicles (EV), or PRP extracellular vesicles, or any combination thereof, is stabilized in a buffered solution having a pH between about 6 and 7. In another embodiment, the pharmaceutical composition comprising topiramate and bioxome, redoxome, HA, extracellular vesicles (EV), or PRP extracellular vesicles, or any combination thereof, is stabilized in a buffered solution having a pH of about 6.5. In another embodiment, the pharmaceutical composition comprising topiramate and bioxome, redoxome, HA, extracellular vesicles (EV), or PRP extracellular vesicles, or any combination thereof, is stabilized in a buffered solution having a pH of about 6.4. In another embodiment, the pharmaceutical composition comprising topiramate and bioxome, redoxome, HA, extracellular vesicles (EV), or PRP extracellular vesicles, or any combination thereof, is stabilized with an amino acid as a stabilizing agent and in some cases a salt (if the amino acid does not contain a charged side chain).

In one embodiment, the pharmaceutical composition comprising a topiramate and bioxome, redoxome, HA, extracellular vesicles (EV), or PRP extracellular vesicles, or any combination thereof, as described herein comprises polymeric microparticles. In another embodiment, the pharmaceutical composition comprising a topiramate and bioxome, redoxome, HA, extracellular vesicles (EV), or PRP extracellular vesicles, or any combination thereof, as described herein comprises nanoparticles. In another embodiment, the pharmaceutical composition comprising a topiramate and bioxome, redoxome, HA, extracellular vesicles (EV), or PRP extracellular vesicles, or any combination thereof, as described herein comprises liposomes. In another embodiment, the pharmaceutical composition comprising a topiramate and bioxome, redoxome, HA, extracellular vesicles (EV), or PRP extracellular vesicles, or any combination thereof, as described herein comprises lipid emulsion. In another embodiment, the pharmaceutical composition comprising a topiramate and bioxome, redoxome, HA, extracellular vesicles (EV), or PRP extracellular vesicles, or any combination thereof, as described herein comprises microspheres. In another embodiment, the pharmaceutical composition comprising a topiramate and bioxome, redoxome, HA, extracellular vesicles (EV), or PRP extracellular vesicles, or any combination thereof, as described herein comprises lipid nanoparticles. In another embodiment, the pharmaceutical composition comprising a topiramate and bioxome, redoxome, HA, extracellular vesicles (EV), or PRP extracellular vesicles, or any combination thereof, as described herein comprises lipid nanoparticles comprising amphiphilic lipids. In another embodiment, the pharmaceutical composition comprising a topiramate and bioxome, redoxome, HA, extracellular vesicles (EV), or PRP extracellular vesicles, or any combination thereof, as described herein comprises lipid nanoparticles comprising a drug, a lipid matrix and a surfactant. In another embodiment, the lipid matrix has a monoglyceride content which is at least 50% w/w.

The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The carrier can be a solvent or dispersion medium containing, for example, water, ethanol, polyol (for example, glycerol, propylene glycol, and liquid polyethylene glycol (PEG), and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, polyalcohols such as mannitol, sorbitol or sodium chloride in the composition. Prolonged absorption of the compositions can be brought about by including in the composition an agent which delays absorption, for example, aluminum monostearate and gelatin.

In some embodiments, the compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

In one embodiment, the composition is provided as a cream. In another embodiment, the composition is provided as an ointment. In another embodiment, the composition is provided as an emulsion. In another embodiment, the composition is provided as a gel base. In another embodiment, the composition is provided as a lotion. In another embodiment, the composition is provided as a paste. In another embodiment, the composition is provided as a foam composition. In another embodiment, the composition is provided as a skin patch. In another embodiment, the composition is provided as a spray.

In some embodiments, the composition is provided as a cetomacrogol emulsifying ointment BP consisting of soft paraffin, liquid paraffin, cetostearyl alcohol and cetomacrogol 1000. This carrier is an oil-in-water base. In some embodiments, the composition is provided as an oil-in-water nanoemulsion consisting of 5-20% oil phase and 80-95 water phase emulsified using phospholipids and non-ionic surfactants.

A skilled artisan would appreciate that ointments are semisolid preparations, typically based on petrolatum or petroleum derivatives. The specific ointment base to be used is one that provides for optimum delivery for topiramate and bioxome, redoxome, HA, extracellular vesicles (EV), or PRP extracellular vesicles, or any combination thereof, and, in some embodiments, provides for other desired characteristics as well (e.g., emolliency). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum.

A skilled artisan would appreciate that emulsion ointment bases are either water-in-oil (W/O) emulsions or oil-in-water (O/W) emulsions, and include, for example, cetyl alcohol, glyceryl monostearate, lanolin and stearic acid. In some embodiments, water-soluble ointment bases are prepared from polyethylene glycols (PEG) of varying molecular weight.

A skilled artisan would appreciate that lotions are preparations that are to be applied to the skin surface without friction. Lotions are typically liquid or semi-liquid preparations in which solid particles, including topiramate and bioxome, redoxome, HA, extracellular vesicles (EV), or PRP extracellular vesicles, or any combination thereof, and present in a water or alcohol base. In some embodiments, lotions disclosed herein may be used for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding topiramate and autologous cells in contact with the skin, such as methylcellulose, sodium carboxymethyl-cellulose, and the like.

In some embodiments, the composition is provided as an aqueous cream formulation comprising water, white soft paraffin, cetosteary alcohol, liquid paraffin and sodium lauryl sulfate. In some embodiments, the composition is provided as a silicon cream formulation comprising water, dimethicone, stearic acid, isopropyl myristate, mineral oil, glycerin, glyceryl stearate, cetyl alcohol, pentenol and TEA. In some embodiments, the composition is provided as an emulsifying ointment aqueous cream comprising soft paraffin, liquid paraffin, cetosterayl alcohol and SLS. This base is oil-in-water emulsion base.

A skilled artisan would appreciate that creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

A skilled artisan would appreciate that pastes are semisolid dosage forms in which topiramate and bioxome, redoxome, HA, extracellular vesicles (EV), or PRP extracellular vesicles, or any combination thereof are suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

In some embodiments, the composition is provided as a topical transparent aqueous gel based on Carbopol polycarboxilic polymers. A skilled artisan would appreciate that gel formulations are semisolid, suspension-type systems. Single-phase gels contain organic macromolecules distributed substantially uniformly throughout the carrier liquid, which is typically aqueous, but in some embodiments, may contain an alcohol and, optionally, an oil. In some embodiments, organic macromolecules, i.e., gelling agents, comprise crosslinked acrylic acid polymers such as the family of carbomer polymers, e.g., carboxypolyalkylenes that may be obtained commercially under the trademark Carbopol™. Other embodiments of polymers in this context comprise hydrophilic polymers such as polyethylene oxides, polyoxyethylene-polyoxypropylene copolymers and polyvinylalcohol.; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A skilled artisan would appreciate that foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropanol. Application and agitation of such a composition at the body temperature cause the isopropanol to vaporize and generate the foam, in a manner similar to a pressurized aerosol foaming system. Foams can be water-based or hydroalcoholic, but are frequently formulated with high alcohol content which, upon application to the skin of a user, quickly evaporates, driving the active ingredient through the upper skin layers to the site of treatment.

A skilled artisan would appreciate that skin patches typically comprise a backing, to which a reservoir containing the active agent is attached. The reservoir can be, for example, a pad in which topiramate and autologous cells or other compositions are dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use skin patches may further comprise a removable cover, which serves for protecting it upon storage.

A skilled artisan would appreciate that sprays provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

In one embodiment, the formulations provided herein also comprise preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents;

viscosity adjustors, such as polymers, including cellulose and derivatives thereof; and polyvinyl alcohol and acid and bases to adjust the pH of these aqueous compositions as needed. The compositions also comprise local anesthetics or other actives. The compositions can be used as sprays, mists, drops, and the like.

In some embodiments, preparation of effective amount or dose can be estimated initially from in vitro/ex vivo assays. In one embodiment, a dose can be formulated in animal models and such information can be used to more accurately determine useful doses in humans.

In some embodiments, toxicity and therapeutic efficacy of the active ingredients described herein can be determined by standard pharmaceutical procedures in vitro, in cell cultures, ex-vivo (human skin) or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p.1].

In one embodiment, depending on the severity and responsiveness of the condition to be treated, dosing can be of a single or a plurality of administrations, with course of treatment lasting from several days to several weeks or until cure is affected or diminution of the disease state is achieved.

In one embodiment, the amount of a composition to be administered will be dependent on the subject being treated, the severity of the affliction, the manner of administration, the judgment of the prescribing physician, etc.

In another embodiment, provided herein is a once weekly dosage form comprising the pharmaceutical formulation provided herein. In another embodiment, provided herein is a once daily dosage form comprising the pharmaceutical formulation provided herein. In another embodiment, provided herein is an every other day dosage form comprising the pharmaceutical formulation provided herein. In another embodiment, provided herein is an every third day dosage form comprising the pharmaceutical formulation provided herein. In another embodiment, provided herein is a twice weekly dosage form comprising the pharmaceutical formulation provided herein. In another embodiment, provided herein is a weekly dosage form comprising the pharmaceutical formulation provided herein. In another embodiment, provided herein is a bi-weekly (every two weeks) dosage form comprising the pharmaceutical formulation provided herein.

In one embodiment, compositions disclosed herein are presented in a pack or dispenser device, such as an FDA approved kit, which contain one or more unit dosage forms containing the active ingredients. In one embodiment, the pack, for example, comprise metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### Methods for Treating

Compositions comprising topiramate and bioxome, redoxome, HA, extracellular vesicles (EV), or PRP extracellular vesicles, or any combination thereof have been described above. Further, therapeutic compositions and pharmaceutical compositions comprising topiramate and bioxome, redoxome, HA, extracellular vesicles (EV), or PRP extracellular vesicles, or any combination thereof have been described above. Those descriptions are incorporated herein in full.

In some embodiments, disclosed herein is a method for treating a dermatological condition comprising a step of administering a composition comprising topiramate and bioxome, redoxome, HA, extracellular vesicles (EV), or PRP extracellular vesicles, or any combination thereof to a subject in need thereof. A skilled artisan would appreciate that, in some embodiments, the terms “dermatological condition”, “dermatological disorder”, “cutaneous condition”, “integumentary condition”, “dermatoses”, “epithelial disorders”, and “dermatological disease” are used herein interchangeably, having all the same qualities and meanings.

In some embodiments, a dermatological condition comprises any medical condition that affects the integumentary system, i.e., the organ system that encloses the body and includes skin, hair, nails, and related muscle and glands. These conditions constitute a broad spectrum of diseases and many nonpathologic states.

In some embodiments, a dermatological condition comprises any disorder that interrupts or causes abnormal growth in epithelial-lined tissue. In some embodiments, epithelium-lined tissue comprises: skin, cornea, lining of organs, or any combination thereof. In some embodiments, dermatological condition comprises a disease affecting the epidermis, the dermis, the subcutaneous tissue, or any combination thereof.

In some embodiments, a dermatological condition comprises cuts. In some embodiments, a dermatological condition comprises scratches. In some embodiments, a dermatological condition comprises wounds. In some embodiments, a dermatological condition comprises incisional wounds. In some embodiments, a dermatological condition comprises excisional wounds. In some embodiments, a dermatological condition comprises sutured wounds. In some embodiments, a dermatological condition comprises glued wounds. In some embodiments, a dermatological condition comprises burns. In some embodiments, a dermatological condition comprises atrophic scars.

In some embodiments, a dermatological condition comprises depressed scars. In some embodiments, a dermatological condition comprises flat scars. In some embodiments, a dermatological condition comprises irregular scars. In some embodiments, a dermatological condition comprises hypertrophic scars. In some embodiments, a dermatological condition comprises keloid scars. In some embodiments, a dermatological condition comprises congenital skin atrophy. In some embodiments, a dermatological condition comprises acute skin atrophy. In some embodiments, a dermatological condition comprises chronic skin diseases. In some embodiments, a dermatological condition comprises inflammatory skin diseases. In some embodiments, a dermatological condition comprises skin infection, skin barrier disorders. In some embodiments, a dermatological condition comprises steroids-derived skin atrophy. In some embodiments, a dermatological condition comprises neoplasm. In some embodiments, a dermatological condition comprises striae.

In some embodiments, a dermatological condition comprises a lesion. In some embodiments, a dermatological condition comprises a primary lesion. In some embodiments, a primary lesion is selected from a group comprising a macule, a patch, a papule, a plaque, a nodule, a tumor, a vesicle, a bulla, a pustule, a cyst, an erosion, an ulcer, a fissure, a weal, a telangiectasia, a burrow, or any combination thereof.

In some embodiments, a dermatological condition comprises a secondary lesion. In some embodiments, a secondary lesion is selected from the group comprising a scale, a crust, a lichenification, an excoriation, an induration, an atrophy, a maceration, an umbilication, a phyma, or any combination thereof.

In some embodiments, a dermatological condition comprises more than a single dermatological condition. For example, a dermatological condition can comprise a keloid scars and a primary lesion.

In some embodiments, a dermatological disorder comprises a wound. A skilled artisan would appreciate that a wound comprises a sharp injury which damages the dermis of the skin. In some embodiments, a wound comprises an open wound, in which the skin is torn, cut, or punctured. In some embodiment, an open wound comprises incisions or incised wounds, lacerations, abrasions, avulsions, puncture wounds, penetration wounds, gunshot wounds, or any combination thereof. In some embodiments, a wound comprises a closed wound, in which a contusion is caused because of trauma. In some embodiments, a closed wound comprises hematomas or crush injury.

In some embodiments, as used herein a wound comprises the wound as well as the area surrounding the wound. In one embodiment, the surrounding area comprises about 1 cm around the wound. In another embodiment, the surrounding area comprises about 2 cm around the wound. In another embodiment, the surrounding area comprises about 3 cm around the wound.

In one embodiment, a wound comprises a chronic wound. In another embodiment, a wound comprises a diabetic ulcer. In another embodiment, a wound comprises pressure sore. In another embodiment, a wound comprises a venous ulcer. In another embodiment, a wound comprises diabetic foot. In another embodiment, a wound comprises a wound associated with aging. In another embodiment, a wound comprises two or more types of wound.

In one embodiment a wound comprises a clean wound, made under sterile conditions and in which no microorganisms are present. In another embodiment a wound comprises a contaminated wound, in which pathogenic microorganisms are present. In another embodiment a wound comprises an infected wound, in which pathogenic microorganisms are present, multiply and clinical symptoms of infection are exhibited. In another embodiment a wound comprises a colonized wound, in which pathogenic microorganism are chronically present.

In some embodiments, provided herein is a composition comprising topiramate and bioxome, redoxome, HA, extracellular vesicles (EV), or PRP extracellular vesicles, or any combination thereof for treating a chronic wound. A skilled artisan would appreciate that a chronic wound is a wound that does not heal in an orderly set of stages and in the time and way that most wounds do. In one embodiment, a chronic wound is a wound that does not heal within three months. Chronic wounds may be detained in one or more of the phases of wound healing. In another embodiment, a chronic wound remains in the inflammatory stage for too long. In another embodiment, a chronic wound remains in the proliferative stage. In another embodiment, a chronic wound is healed by diminishing bacterial burden, necrotic tissue, inflammation, or any combination thereof.

In some embodiments, a dermatological condition comprises a scar. A skilled artisan would appreciate that a scar comprises an area of fibrous tissue that replaces normal skin after an injury. Scars result from the natural process of wound repair in the skin, or in other tissues of the body. Though scar tissue is composed of the same proteins as the tissue that it replaces, the fiber composition of the protein is different. In a scar, the collagen cross-links and forms a pronounced alignment in a single direction. This tissue alignment is usually of inferior functional quality to the normal collagen randomized alignment. For example, scars in the skin are less resistant to ultraviolet radiation and sweat glands and hair follicles do not grow back within scar tissues.

In one embodiment, a scar comprises a chronic scar. In another embodiment, a scar comprises a post-acne scar. In another embodiment, a scar comprises a post-varicella scar. In another embodiment, a scar comprises post dermatitis pigmentation. In another embodiment, a scar comprises a post-traumatic scar. In another embodiment, a scar comprises a hypertrophic scar. In another embodiment, a scar comprises keloids. In another embodiment, a scar comprises melisma. In some embodiments, a scar comprises two or more types of scar.

In one embodiment, treating a wound or a scar comprises healing said wound or said scar. In another embodiment, treating a wound or a scar comprises reducing the size of said wound or said scar. In another embodiment, treating a wound or a scar comprises enhancing the vascularization in said wound or said scar. In another embodiment, treating a wound or a scar comprises diminishing the exudate in said wound or said scar. In another embodiment, treating a wound or a scar comprises reducing necrosis of said wound or said scar. In another embodiment, treating a wound or a scar comprises reducing microorganisms in said wound or said scar. In another embodiment, treating a wound or a scar comprises reducing pain in said wound or said scar. In another embodiment, treating a wound or a scar comprises reducing scar tissue in said wound or said scar. In another embodiment, treating a wound or a scar comprises preventing scar tissue in said wound or said scar. In another embodiment, treating a wound or a scar comprises accelerating the closure of said wound or said scar.

A skilled artisan would appreciate that wound or scar healing is a complex process in which the skin and the affected tissues are repaired. Physiologically, wound healing comprises a regulated sequence of biochemical events set into motion to repair the damage. These biochemical events include: hemostasis, inflammation, proliferation of a new tissue, and remodeling. In one embodiment, administration of a composition comprising topiramate and bioxome, redoxome, HA, extracellular vesicles (EV), or PRP extracellular vesicles, or any combination thereof regulates hemostasis. In another embodiment, administration of a composition comprising topiramate and bioxome, redoxome, HA, extracellular vesicles (EV), or PRP extracellular vesicles, or any combination thereof regulates inflammation. In another embodiment, administration of a composition comprising topiramate and bioxome, redoxome, HA, extracellular vesicles (EV), or PRP extracellular vesicles, or any combination thereof regulates proliferation of new tissue. In another embodiment, administration of a composition comprising topiramate bioxome, redoxome, HA, extracellular vesicles (EV), or PRP extracellular vesicles, or any combination thereof regulates tissue remodeling.

A skilled artisan would appreciate that the term “treating” encompasses both therapeutic treatment and prophylactic or preventative measures, wherein the object is to prevent or lessen the targeted pathologic condition or disorder as described hereinabove.

Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” may encompass, inter alia, to delaying progression, expediting remission, inducing remission, augmenting remission, speeding recovery, increasing efficacy of or decreasing resistance to alternative therapeutics, or a combination thereof. In one embodiment, “preventing” may encompass, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, may encompass, inter alia, to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments, the results of use of a composition described herein provides unexpected improved treatment compared with use of any of the elements comprised in the composition and used independently. In some embodiments, the results of use of a composition described herein produces a synergistic effect wherein the result of use produces a combined effect greater than the sum of their separate effects.

In one embodiment, administration of a composition described herein comprises concurrent administration of topiramate and bioxome, redoxome, HA, extracellular vesicles (EV), or PRP extracellular vesicles, or any combination thereof. In another embodiment, administration of a combination of topiramate and bioxome, redoxome, HA, extracellular vesicles (EV), or PRP extracellular vesicles, or any combination thereof comprises administration of at least one composition comprising at least one component prior to the administration of another composition comprising at least a different component. In another embodiment, administration of a combination of topiramate and bioxome, redoxome, HA, extracellular vesicles (EV), or PRP extracellular vesicles, or any combination thereof or composition(s) thereof comprises administration all compositions concurrently. In another embodiment, administration of a combination of topiramate and bioxome, redoxome, HA, extracellular vesicles (EV), or PRP extracellular vesicles, or any combination thereof or composition(s) thereof comprises administration of at least one composition comprising at least one component following the administration of another composition comprising at least a different component.

In one embodiment, an administration of a composition comprises a single administration. In another embodiment, an administration of a composition comprises a follow-up administration. In another embodiment, an administration of a composition comprises multiple follow-up administrations.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

### Ex-Vivo Model

Though inflammation is a common feature of several skin diseases including psoriasis, atopic dermatitis, seborrheic dermatitis and contact dermatitis, the characteristics of the cellular immune response and composition of cytokine profile vary among them. Several in vitro, ex vivo human skin explants and in vivo models mimic the inflammatory response of the skin.

Ex vivo skin explants can be used to monitor and screen different aspects of cutaneous disorders, including UVB-induced damage, oxidative stress, skin irritation, skin inflammation and functional assays. Ex vivo system is being used for anti-inflammatory screening and for the investigation of the complex interplay between the regulatory mechanisms of cytokine secretion.

Cell cultures, stimulated by lipopolysaccharide (LPS) or 12-O-tetradecanoylphorbol-13-acetate (TPA), are routinely used methods for both gaining basic knowledge of the molecular pathways and regulation of the immune response, and as models for evaluating anti-inflammatory properties of novel agents.

In the examples describes below, ex-vivo human skin explants are treated with or without lipopolysaccharide (LPS) and the impact on the pro-inflammatory cytokine TNF-αlpha secretion is evaluated.

Lipopolysaccharide (LPS) is a well-known pathogen-associated molecular pattern, primarily found on the outer leaflet of the external membrane in most gram-negative bacteria. Although the LPS molecule varies among different organisms, especially in the length and fatty acid composition of the lipid A domain, the basic structure is mostly preserved. LPS induces its main effect by activation of the Toll-like receptor (TLR) family members, which are predominant pattern-recognition receptors.

### Example 1: Epidermal Viability of the Ex Vivo Organ Cultures of Human Skin

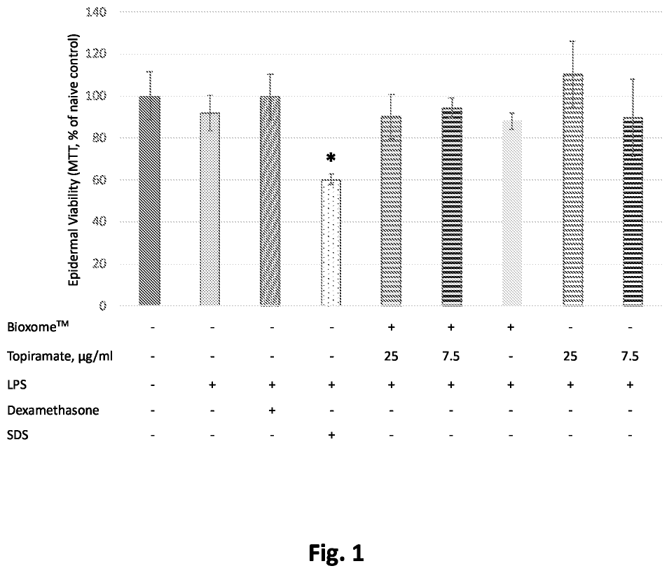

Ex vivo organ cultures of human skin were challenged with 5 mg/ml of LPS and cultured for 24 h in skin culture medium containing various doses of Topiramate, alone or encapsulated in Bioxome™. Empty Bioxome control was used. Throughout the study, similar concentration of Bioxome™ was used. Dexamethasone (10 mM) was used as a positive anti-inflammatory control. SDS (10%) was used as positive viability control (cell death inducer). At the end of the experiment, viability of the skin explants was assessed by MTT assay. MTT data in all treatment groups were normalized to the naïve skin control group (No treatment). Data are presented in FIG. 1 as mean+SD from 3 independent skin cultures and analyzed by Student's t-test; values differ from Naive control: * P-value<0.005.

### Example 2: Anti-Inflammatory Effect of Bioxome™-Encapsulated Topiramate in the Ex Vivo Human Skin Inflammation Model

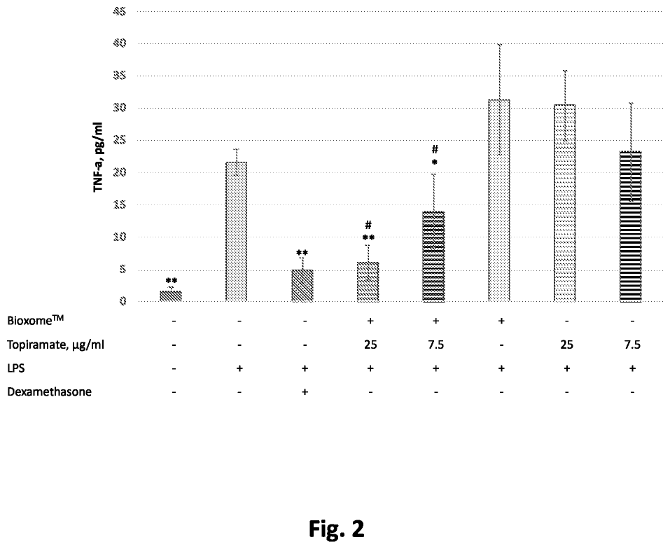

Ex vivo organ cultures of human skin were challenged with 5 mg/ml of LPS and cultured for 24 h in skin culture medium containing various doses of Topiramate, alone or encapsulated in Bioxome™. Empty Bioxome control was used. Throughout the study, same concentration of Bioxome™ was used. Dexamethasone (10 mM) was used as positive control. At the end of the experiment, the accumulation of TNF-α was measured in supernatants. As is evident from the data presented in FIG. 2, topiramate encapsulated in Bioxome™ surprisingly presents a superior anti-inflammatory effect, in comparison to topiramate alone or Bioxome™ alone. Data is presented as mean+SD concentration per milliliter of supernatant from 3 independent skin cultures and analyzed by Student's t-test; values differ from inflammation control (LPS only): * P-value<0.05, **P value<0.002; Values do not differ from positive control (LPS with Dexamethasone): # P-value>0.05

### Example 3: Anti-Inflammatory Raw Cells

The effect of Topiramate with and without Bioxome on the secretion of several anti-inflammatory markers including interleukin (IL)-12, IL-17, IL-22, IL-23, IL-6, IL-8, IL-17 and nitric oxide is determined in macrophage RAW 264.7 cells.

The cells are treated with or without four concentrations of each Test item for 24 hr, and stimulated with LPS or with IL-17. Cell viability is determined by MTT in triplicates.

Concomitantly, cytokines secretion levels are evaluated in two selected concentrations of each Test item.

The compatibility and possible synergistic action are evaluated for 30 combinations for each inflammatory marker.

### Example 4: Epidermis Turnover Rate: BrdU

The effect of Topiramate with and without Bioxome on cell proliferation is measured. Cell proliferation is measured by BrdU method that determines the ability of test items to reduce their proliferation rate in vitro or ex vivo.

BrdU is a synthetic nucleoside, an analog of thymidine. BrdU can be incorporated into synthesized DNA of replicating cells (S phase). Reduction in BrdU=reduction in proliferation. The possible synergistic action in inhibiting cell proliferation is evaluated.

### Example 5: Histological Evaluation

To ascertain the impact of Topiramate with and without Bioxome on inflammation-induced hyperproliferation and skin morphology, histological examination is performed on ex-vivo human skin stimulated with LPS/EGF and/or IL-17 stimulation. Histological evaluation of H&E-stained slides Ki67 immunohistochemistry (proliferation) and psoriasin is performed.

A possible synergistic effect of the two compounds with and without final formulation is measured.

### Example 6: Anti-Inflammatory: Keratinocytes Following UV and Anti-Oxidant Effect

The impact of Topiramate with and without Bioxome on the UV-B induced secretion of prostaglandin E2 (PGE2), IL-6, IL-8 is determined in keratinocytes HaCaT cells.

The cells are treated with or without four concentrations of each Test item for 24 hr and stimulated with UV radiation. Cell viability is determined by MTT in triplicates.

Concomitantly, cytokines secretion levels will be evaluated in two selected concentrations of each Test item.

In addition, a similar set stimulated by glucose oxidase is monitored in order to obtain the cellular antioxidant capacity of the compounds. Similarly, the compatibility and possible synergistic action are evaluated for 30 combinations.

### Example 7: MMP and Hyaluronic Acid Synthesis

The effect of Topiramate with and without Bioxome on MMP-1 (collagenase) activity is measured by its ability to cleave specific substrate to form a fluorescent product using appropriate commercial kits. Calibration curve is generated in duplicates according to the manufacturer's instructions. Concomitantly, Hyaluronic acid synthesis are determined by an appropriate commercial kit, according to the manufacturer's instructions. Possible synergistic action is evaluated for 30 combinations.

### Example 8: Histological Evaluation

Ex-vivo histological evaluation on human skin treated with Topiramate with or without Bioxome and/or final formulations containing Topiramate with and without Bioxome is measured following UV radiation.

Changes in elastin and collagen are measured to evaluate photo-aging effects.

A possible synergistic effect of the two compounds with and without final formulation is measured.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising topiramate and bioxome, redoxome, Hyaluronic acid (HA), extracellular vesicles (EV), or platelet rich derived (PRP) extracellular vesicles, or any combination thereof.

2. The composition of claim 1, comprising topiramate and a bioxome.

3. The composition of claim 2, wherein said topiramate is encapsulated by said bioxome.

4. The composition of claim 1, further comprising an excipient.

5. The composition of claim 4, wherein at least one excipient, or topiramate, or any combination thereof is encapsulated in said bioxome, in said redoxome, in said HA, in said extracellular vesicles (EV), or in said PRP extracellular vesicles.

6. The composition of claim 1, wherein said composition is formulated for topical delivery.

7. The composition of claim 6, further comprising an excipient selected from the group comprising: C12-C15 alkyl benzoate, mineral oil, glycerin, cetearyl alcohol, glyceryl stearate SE, dimethicone, steareth-2, steareth-21, aloe barbadensis leaf juice, panthenol, disodium EDTA, acrylates/C10-30 alkyl acrylate crosspolymer, tocopheryl acetate, retinyl palmitate, hydroxypropyl cyclodextrin, palmitoyl tripeptide 38, hydroxyethilcellulose, sodium hydroxide, phenoxyethanol, caprylyl glycol, or any combination thereof.

8. The composition of claim 1, further comprising an additional therapeutic agent for healing inflammatory condition of the skin.

9. The compositions of claim 8, wherein said additional therapeutic agent is selected from a group comprising Non Steroidal Anti Inflammatory Drugs (NSAIDs), antileukotrienes, Immune Selective Anti-Inflammatory Derivatives (ImSAIDs), anti TNFalpha drugs, or any combination thereof.

10. A method for treating a dermatological condition comprising a step of administering to a subject in need thereof a composition comprising topiramate and bioxome, redoxome, HA, extracellular vesicles (EV), or PRP extracellular vesicles, or any combination thereof.

11. The method of claim 10, wherein said dermatological condition is an inflammatory condition.

12. The method of claim 11, wherein said inflammatory condition is a TNF mediated inflammatory disease, selected from skin aging or psoriasis.

13. (canceled)

14. (canceled)

15. (canceled)

16. The method of claim 10, wherein said composition comprises topiramate and a bioxome.

17. The method of claim 16, wherein said topiramate is encapsulated by said bioxome.

18. The method of claim 10, wherein said composition further comprises an excipient.

19. The method of claim 10, wherein at least one excipient, or topiramate, or any combination thereof is encapsulated in said bioxome, in said redoxome, in said HA, in said extracellular vesicles (EV), or in said PRP extracellular vesicles.

20. The method of claim 10, wherein said composition is administered topically.

21. The method of claim 20, wherein said composition further comprising an excipient selected from the group comprising: C12-C15 alkyl benzoate, mineral oil, glycerin, cetearyl alcohol, glyceryl stearate SE, dimethicone, steareth-2, steareth-21, aloe barbadensis leaf juice, panthenol, disodium EDTA, acrylates/C10-30 alkyl acrylate crosspolymer, tocopheryl acetate, retinyl palmitate, hydroxypropyl cyclodextrin, palmitoyl tripeptide 38, hydroxyethilcellulose, sodium hydroxide, phenoxyethanol, caprylyl glycol, or any combination thereof.

22. The method of claim 10, wherein said composition further comprises an additional therapeutic agent.

23. (canceled)

24. The method of claim 22 wherein said additional therapeutic agent is selected from a group comprising Non Steroidal Anti Inflammatory Drugs (NSAIDs), antileukotrienes, Immune Selective Anti-Inflammatory Derivatives (ImSAIDs), anti TNFalpha drugs, or any combination thereof.
